(19) United States
(12) Patent Application Publication
(10) Pub. No.: US 2005/0169946 A1
(43) Pub. Date: Aug. 4, 2005

(54) Cosmetic formulation comprising at least two active substances in a multiple emulsion optionally mixed with a single emulsion

(76) Inventors: Helene Lannibois, Charenton Le Pont (FR); Jean-Marc Ricca, Orchard Bel-Air (SG)

Correspondence Address: Jean Louis Seugnet;Rhodia Inc CN 7500, Intellectual Property Department, 259 Prospect Plains Road, Cranbury, NJ, 08512-7500, US

(21) Appl. No.: 10/506,380

(22) PCT Filed: Mar. 4, 2002
(86) PCT No.: PCT/FR02/00769

(51) Int. Cl. A61K 7/00

(52) U.S. Cl. 424/401

(57) ABSTRACT

The invention concerns a cosmetic and/or dermatological composition comprising at least two active substances in a multiple emulsion consisting of an internal aqueous phase dispersed in an internal oily phase, the whole being dispersed in an external aqueous phase, the multiple emulsion being optionally mixed with a single emulsion consisting of an external oily phase dispersed in an external aqueous phase: the internal oily phase comprising at least one nonionic surfactant and/or at least one amphiphilic polymer and optionally at least one hydrophobic active substance; the external aqueous phase comprising at least one nonionic surfactant and/or at least one nonionic amphiphilic polymer optionally associated with at least one anionic amphiphilic polymer or comprising at least one anionic amphiphilic polymer optionally associated with at least one anionic surfactant; at least one hydrophilic active substance being present in the internal aqueous phase; and at least one active substance being present in soluble form, solubilized or in the form of a solid dispersed in the external aqueous phase, or being present in the external oily phase.

The subject of the present invention is cosmetic and/or dermatological formulations comprising at least two active materials in a multiple emulsion of the water-in-oil-in-water type, optionally mixed with a simple emulsion of the oil-in-water type.

It is increasingly being offered to consumers to have, with the same formulation, a plurality of simultaneous and/or successive effects. Such performances are obtained by combining in the formulation several active substances.

This poses no major difficulty if the active materials are compatible with each other and with the formulation medium in which they are introduced.

In case of incompatibility, it has then been envisaged to encapsulate one of the active materials in order to isolate it from the medium and/or from the other active materials, or alternatively in order to delay the effect of said encapsulated active material; the encapsulated material is then dispersed in the medium. By way of example of a solution of this type, perfumes have been encapsulated into polymeric latexes. However, even if these methods offer real advantages, they are nevertheless limited to particular active materials. Indeed, these methods are hardly applicable to the encapsulation of hydrophilic active materials. Furthermore, if the active material is solid, it is necessary to find a diluent which is a swelling compound of said latex without solubilizing it. Needless to specify that with applications in the cosmetics field, this choice is made all the more difficult since the diluent which can be selected must be approved in this field.

Thus, as can be observed, there is currently no satisfactory, simple and effective means of using at least two active materials which it is desired to isolate from each other and/or from the medium into which they are introduced.

These aims and others are achieved by the present invention which therefore consists of a cosmetic and/or dermatological formulation comprising at least two active materials in a multiple emulsion consisting of an internal aqueous phase dispersed in an internal oily phase, the whole being dispersed in an external aqueous phase; the multiple emulsion being optionally mixed with a simple emulsion consisting of an external oily phase dispersed in an external aqueous phase:
the internal oily phase comprising at least one nonionic surfactant and/or at least one amphiphilic polymer and optionally at least one hydrophobic active material;
the external aqueous phase comprising at least nonionic surfactant and/or at least one nonionic amphiphilic polymer optionally combined with at least one anionic amphiphilic polymer or comprising at least one anionic amphiphilic polymer optionally combined with at least one anionic surfactant;
at least one hydrophilic active material which is present in the internal aqueous phase; and
at least one active material which is present in a soluble or solubilized form or in the form of a solid dispersed in the external aqueous phase, or which is present in the external oily phase.

It is pointed out that in the description which follows, the term mixed emulsion will denote either the multiple emulsion, or the multiple emulsion mixed with the simple emulsion.

Thus, the mixed emulsion entering into the composition of the cosmetic and/or dermatological formulations has the advantage of being easy to prepare and to use, without requiring substantial investments.

It additionally makes it possible to isolate any type of liquid or solid, hydrophobic or hydrophilic, active material.

The mixed emulsion is most particularly appropriate when two active materials, which are advantageously incompatible, are used, one being hydrophilic, the other hydrophobic; the first being introduced into the internal aqueous phase and the second into the external oily phase. In this way, the two active materials are isolated from each other in the mixed emulsion.

Furthermore, a delayed effect can be observed during the application of the formulation, for the least available active material, for example the one present in the internal aqueous phase of the mixed emulsion.

However, other aims and advantages will appear more clearly on reading the description and the example which follow.

In the description, the term polymer denotes both homopolymers and copolymers.

For the sake of simplicity, in the disclosure of the invention, the inverse emulsion of the multiple emulsion and its mode of preparation will first of all be detailed.

The inverse emulsion therefore consists of a water-in-oil emulsion consisting of an internal aqueous phase and an internal oily phase.

The internal oily phase comprises at least one organic oil, of animal or plant origin, or mineral oil, and waxes obtained from the same origins, or mixtures thereof.

Preferably, an oily phase is used which is fluid under the conditions for preparing the inverse emulsion.

The compound used as oily phase is preferably chosen from compounds whose solubility in water does not exceed 10% by weight at 25° C.

As organic oils of animal origin, there may be mentioned, inter alia, cachalot oil, whale oil, seal oil, sardine oil, herring oil, shark oil, cod liver oil; lard, mutton fat (tallow).

As waxes of animal origin, beeswax may be mentioned.

As examples of organic oils of plant origin, there may be mentioned, inter alia, rapeseed oil, sunflower oil, peanut oil, olive oil, walnut oil, corn oil, soybean oil, linseed oil, hemp oil, grapeseed oil, copra oil, palm oil, cottonseed oil, babassu oil, jojoba oil, sesame oil and castor oil.

As waxes of plant origin, carnauba wax may be mentioned.

As regards mineral oils, there may be mentioned, inter alia, naphthenic oils or paraffin oils (petroleum jelly). Paraffin waxes may likewise be suitable for preparing the emulsion.

The products derived from alcoholysis of the abovementioned oils may also be used.

There would be no departure from the scope of the present invention by using, as internal oily phase, at least one saturated or unsaturated fatty acid, at least one saturated or unsaturated fatty acid ester, at least one saturated or unsaturated fatty alcohol, or mixtures thereof.

More particularly, said acids, esters or alcohols comprise at least one hydrocarbon radical having from 10 to 40 carbon atoms, more particularly 18 to 40 carbon atoms, and may comprise one or more conjugated or nonconjugated carbon-carbon double bonds. Moreover, the acids, esters or alcohols may comprise one or more hydroxyl groups.

As examples of saturated fatty acids, there may be mentioned palmitic, stearic and behenic acids.

As examples of unsaturated fatty acids, there may be mentioned myristoleic, palmitoleic, oleic, erucic, linoleic, linolenic, arachidonic and ricinoleic acids, and mixtures thereof.

As fatty acid esters, there may be mentioned the esters of the acids listed above, for which the part derived from the alcohol comprises 1 to 6 carbon atoms, such as methyl, ethyl, propyl and isopropyl esters, and the like.

As examples of alcohols, there may be mentioned those corresponding to the abovementioned acids.

It is likewise possible to envisage using the esters of the acids mentioned above and of polyols, such as for example of glycerol, polyglycerol (such as for example polyglyceryl polyricinoleate), glycol, propylene glycol, ethylene glycol, polyethylene glycol, polypropylene glycol, neopentyl glycol (such as for example neopentyl glycol hydroxypivalate), pentaerythritol, dipentaerythritol, trimethylolpropane, sorbitol, mannitol, xylitol and mesoerythritol.

The internal oily phase may likewise be chosen from essential oils, mono-, di- and triglycerides, and silicone oils.

It may comprise at least one hydrophobic active material, as long as it is compatible with the hydrophilic active material present in the internal aqueous phase and, if it is present, in the external aqueous phase; the phases will be described later.

Said hydrophobic active materials exist in liquid form, solubilized in an organic solvent, or alternatively in the form of a divided solid dispersed in said phase.

More particularly, the active materials are such that their solubility in water does not exceed 10% by weight, at 25° C.

The active materials whose melting point is less than or equal to 100° C., more particularly less than or equal to 80° C., may likewise be used.

By way of examples of active materials which can be used in the cosmetics field, there may be mentioned the silicone oils belonging for example to the dimethicone family; lipophilic vitamins, such as vitamin A and its derivatives, in particular its esters such as the acetate, palmitate or propionate, vitamin B2, pantothenic acid, vitamin D and vitamin E; mono-, di- and triglycerides; bactericides; UV absorbers, such as aminobenzoate derivatives of the PABA and PARA type, salicylates, cinnamates, anthranilates, dibenzoyl-methanes, camphor derivatives and mixtures thereof.

Antiaging agents may likewise be used. As examples of such agents, there may be mentioned in particular retinoids, fat-soluble vitamins, derivatives of vitamin C such as the esters, in particular the acetate, propionate or palmitate; ceramides, pseudoceramides, phospholipids, fatty acids, fatty alcohols, cholesterol, sterols and mixtures thereof. As preferred fatty acids and alcohols, there may be mentioned more particularly those possessing linear or branched alkyl chains containing from 12 to 20 carbon atoms. This may be in particular linoleic acid.

It is likewise possible to use anticellulite agents such as in particular isobutylmethylxanthine and theophylline; and antiacne agents such as for example resorcinol, resorcinol acetate, benzoyl peroxide and numerous natural compounds.

Flavorings, perfumes, essential oils and essences may also be used as hydrophobic active material. By way of example, there may be mentioned oils and/or essences of mint, spearmint, peppermint, menthol, vanilla, cinnamon, bay tree, anise, eucalyptus, thyme, sage, cedar leaf, nutmeg, citrus (lemon, lime, grapefruit, orange), fruits (apple, pear, peach, cherry, plum, strawberry, raspberry, apricot, pineapple, grape, and the like), alone or as mixtures. It is also possible to use compounds such as benzaldehyde, isoamyl acetate, ethyl butyrate, and the like.

The antimicrobial agents may be chosen from thymol, menthol, triclosan, 4-hexylresorcinol, phenol, eucalyptol, benzoic acid, benzoic peroxide, butylparaben, and mixtures thereof.

It should be noted that it is not impossible for the oily phase itself to be considered as hydrophobic active material.

In the case where the internal oily phase comprises one or more different hydrophobic active materials of the oily phase, their content represents more particularly 10 to 50% by weight of said internal oily phase.

The inverse emulsion additionally comprises at least one nonionic surfactant and/or at least one amphiphilic, preferably block, polymer.

It should be noted that the Bancroft rule may be applied to the nonionic surfactant and to the amphiphilic polymer, preferably block polymer, used (2nd World Conference on Emulsion, 1997, Bordeaux, France). In other words, the fraction soluble in the continuous phase is greater than the fraction soluble in the dispersed phase.

Thus, the surfactant and the polymer are preferably chosen from those which simultaneously satisfy of the two conditions below:
when they are mixed with the internal oily phase, at a concentration between 0.1 and 10% by weight of said phase at 25° C., they are in the form of a solution in the whole or part of the concentration range indicated;
when they are mixed with the internal aqueous phase, at a concentration between 0.1 and 10% by weight of said phase and at 25° C., they are in the form of a dispersion in the whole or part of the concentration range indicated.

More particularly, the nonionic surfactant is chosen from compounds having an HLB (hydrophilic/lipophilic balance) value of less than or equal to 8.

By way of examples of surfactants which may enter into the composition of the inverse emulsion, there may be mentioned surfactants, alone or in the form of a mixture, chosen from:
alkoxylated fatty alcohols
alkoxylated triglycerides
alkoxylated fatty acids
optionally alkoxylated sorbitan esters
alkoxylated fatty amines
alkoxylated di(1-phenylethyl)phenols
alkoxylated tri(1-phenylethyl)phenols
alkoxylated alkylphenols
   the number of alkoxylated (ethoxylated, propoxylated, butoxylated) units is such that the HLB value is less than or equal to 8.

The alkoxylated fatty alcohols generally comprise from 6 to 22 carbon atoms, the alkoxylated units being excluded from these numbers.

The alkoxylated triglycerides may be triglycerides of plant or animal origin.

The optionally alkoxylated sorbitan esters are cyclized sorbitol esters of fatty acids comprising from 10 to 20 carbon atoms, such as lauric acid, stearic acid or oleic acid.

The alkoxylated fatty amines generally have from 10 to 22 carbon atoms, the alkoxylated units being excluded from these numbers.

The alkoxylated alkylphenols generally have one or two linear or branched alkyl groups having 4 to 12 carbon atoms. By way of example, there may be mentioned in particular octyl, nonyl or dodecyl groups.

As for the amphiphilic polymer, it advantageously comprises at least two blocks.

These amphiphilic polymers, which satisfy the Bancroft rule and the two conditions set out above, comprise more particularly at least one hydrophobic block and at least one neutral or anionic hydrophilic block.

In the case where the amphiphilic polymer comprises at least three blocks, and more particularly three blocks, the polymer is preferably linear. In addition, the hydrophobic blocks are more particularly at the ends.

In the case where the polymers comprise more than three blocks, the latter are preferably in the form of graft or comb polymers.

In the text which follows, even though this constitutes a misuse of language, the term amphiphilic block polymer will be used without distinction for linear block polymers and graft or comb polymers.

Said amphiphilic polymers may advantageously be obtained by the so-called live or controlled free-radical polymerization. By way of nonlimiting examples of methods of so-called live or controlled polymerization, reference may be made in particular to applications WO 98/58974 (xanthate), WO 98/01478 (dithioesters), WO 99/03894 (nitroxides); WO 99/31144 (dithiocarbamates).

The amphiphilic polymers may also be obtained by anionic polymerization.

They may likewise be prepared using (in particular anionic or cationic) ring opening polymerizations, or by chemical modification of the polymer.

The graft or comb polymers may also be obtained by so-called direct grafting and copolymerization methods.

Direct grafting consists in polymerizing the chosen monomer(s) by the free-radical route, in the presence of the selected polymer to form the backbone of the final product. If the monomer/backbone pair and the operating conditions are judiciously chosen, then a transfer reaction may take place between the growing macroradical and the backbone. This reaction generates a radical on the backbone, and it is from this radical that the graft grows. The primary radical obtained from the initiator may also contribute to the transfer reactions.

As regards the copolymerization, it uses, in a first instance, grafting to the end of the future pendent segment a functional group which can be polymerized by the free-radical route. This grafting may be carried out by customary methods of organic chemistry. Next, in a second instance, the macromonomer thus obtained is polymerized with the chosen monomer to form the backbone and a so-called “comb” polymer is obtained.

Among the hydrophobic monomers from which the hydrophobic blocks of the amphiphilic polymer may be prepared, there may be mentioned in particular:
esters of linear, branched, cyclic or aromatic, mono- or polycarboxylic acids comprising at least one ethylenic unsaturation,
esters of saturated carboxylic acids preferably comprising 8 to 30 carbon atoms, optionally carrying a hydroxyl group;
αβ-ethylenically unsaturated nitrites, vinyl ethers, vinyl esters, vinylaromatic monomers, vinyl or vinylidene halides,
linear or branched, aromatic or nonaromatic hydrocarbon monomers comprising at least one ethylenic unsaturation,
monomers of the cyclic or noncyclic siloxane type, chlorosilanes;
propylene oxide, butylene oxide;
   alone or in the form of mixtures, and macromonomers derived from such monomers.

By way of particular examples of hydrophobic monomers which may enter into the preparation of the hydrophobic block(s) of the amphiphilic block polymer, there may be mentioned:
esters of (meth)acrylic acid with an alcohol comprising 1 to 12 carbon atoms such as methyl (meth)acrylate, ethyl (meth)acrylate, propyl (meth)acrylate, n-butyl (meth)acrylate, t-butyl (meth)acrylate, isobutyl (meth)acrylate, 2-ethylhexyl acrylate;
vinyl acetate, vinyl Versatate®, vinyl propionate, vinyl chloride, vinylidene chloride, methyl vinyl ether, ethyl vinyl ether;
the vinyl nitriles include more particularly those having from 3 to 12 carbon atoms, such as in particular acrylonitrile and methacrylonitrile;
styrene, α-methylstyrene, vinyltoluene, butadiene, isoprene, chloroprene;
   alone or in the form of mixtures, and the macromonomers derived from such monomers.

The preferred monomers are esters of acrylic acid with linear or branched C₁-C₄ alcohols such as methyl, ethyl, propyl and butyl acrylate, vinyl esters such as vinyl acetate, styrene, α-methylstyrene.

As regards the nonionic hydrophilic monomers from which the amphiphilic block polymers may be obtained, there may be mentioned, with no limitation being implied, ethylene oxide, amides of linear, branched, cyclic or aromatic mono- or polycarboxylic acids comprising at least one ethylenic unsaturation or derivatives, such as (meth)acrylamide, N-methylol (meth)acrylamide; hydrophilic esters derived from (meth)acrylic acid such as for example 2-hydroxyethyl (meth)acrylate; vinyl esters which make it possible to obtain polyvinyl alcohol blocks after hydrolysis, such as vinyl acetate, vinyl Versatate®, vinyl propionate, alone or in combination, and macromonomers derived from such monomers. It is recalled that the term macromonomer denotes a macromolecule carrying one or more polymerizable functional groups.

However, the preferred hydrophilic monomers are acrylamide and methacrylamide, alone or in the form of a mixture, or in the form of macromonomers.

As regards the anionic hydrophilic monomers from which the amphiphilic block polymers may be obtained, there may be mentioned, for example, monomers comprising at least one carboxylic, sulfonic, sulfuric, phosphonic, phosphoric, or sulfosuccinic functional group, or the corresponding salts.

It is specified that under the pH conditions for using the amphiphilic block polymer, the functional groups of the anionic block(s) of the polymer are in a form which is at least partially ionized (dissociated). More particularly, at least 10 mol % of the functional groups of the block(s) are in ionized form. The determination of this value does not pose any problem for persons skilled in the art; it depends in particular on the pKa of the ionizable functional groups of the units of the polymer and on the number of these functional groups (that is on the number of mol of monomer carrying ionizable functional groups used during the preparation of the polymer).

More particularly, the monomers are chosen from:
linear, branched, cyclic or aromatic mono- or polycarboxylic acids, N-substituted derivatives of such acids; monoesters of polycarboxylic acids, comprising at least one ethylenic unsaturation;
linear, branched, cyclic or aromatic vinylcarboxylic acids;
amino acids comprising one or more ethylenic unsaturations;
   alone or in the form of mixtures, their precursors, their sulfonic or phosphonic homologs, and the macromonomers derived from such monomers; it being possible for the monomers or macromonomers to be in the form of salts.

By way of examples of anionic monomers, there may be mentioned without limitation being implied:
acrylic acid, methacrylic acid, fumaric acid, itaconic acid, citraconic acid, maleic acid, acrylamido glycolic acid, 2-propene-1-sulfonic acid, methallylsulfonic acid, styrenesulfonic acid, α-acrylamidomethylpropanesulfonic acid, 2-sulfoethylene methacrylate, sulfopropylacrylic acid, bis-sulfopropylacrylic acid, bis-sulfopropylmethacrylic acid, sulfatoethyl-methacrylic acid, phosphate monoester of hydroxyethyl methacrylic acid, and the alkali metal salts, such as sodium or potassium, or ammonium salts;
vinylsulfonic acid, vinylbenzenesulfonic acid, vinylphosphonic acid, vinylidenephosphoric acid, vinylbenzoic acid, and the alkali metal salts, such as sodium or potassium, or ammonium salts;
N-methacryloylalanine, N-acryloylhydroxyglycine;
   alone or in the form of mixtures, and the macromonomers derived from such monomers.

There would be no departure from the scope of the present invention in using monomers which are precursors of those which have just been cited. In other words, these monomers have units which, once incorporated into the polymer chain, may be converted, in particular by chemical treatment such as hydrolysis, to give again the abovementioned anionic species. For example, the completely or partially esterified monomers of the abovementioned monomers may be used so as to be subsequently completely or partially hydrolyzed.

Preferably, the amphiphilic block polymers have a weight-average molar mass of less than or equal to 100 000 g/mol, more particularly between 1 000 and 50 000 g/mol, preferably between 1 000 and 20 000 g/mol. It is specified that the weight-average molar masses indicated above are theoretical molar masses evaluated according to the respective quantities of the monomers introduced during the preparation of said polymers.

Preferably, an amphiphilic block polymer of the nonionic type is used.

By way of example of an amphiphilic block polymer suitable for carrying out the invention, there may be mentioned polyhydroxystearate—polyethylene glycol—polyhydroxystearate triblock polymers (the products in the Arlacel range from ICI are an example thereof), polyether polyalkyl graft polydimethylsiloxane block polymers (such as the products of the trademark Tegopren marketed by Goldschmidt).

Preferably, at least one surfactant, at least one nonionic amphiphilic polymer, or mixtures thereof is (are) used.

According to a particularly advantageous embodiment, the inverse emulsion comprises one amphiphilic, preferably block and nonionic, polymer, or a mixture of several of them.

The total quantity of surfactant and/or amphiphilic polymer preferably represents from 2 to 10% by weight of the internal oily phase.

The internal aqueous phase comprises at least one hydrophilic active material, provided in a form soluble in the internal aqueous phase; in a form solubilized in a water-miscible solvent such as methanol, ethanol, propylene glycol, glycerol; in the form of a divided solid dispersed in said phase.

The content of hydrophilic active material is more particularly between 0.1 and 50% by weight of the internal aqueous phase, and preferably between 0.1 and 20% by weight of the internal aqueous phase.

By way of examples of active materials which may be used in the cosmetic field, there may be mentioned substances which have a cosmetic effect, a therapeutic effect or any other substance which may be used for treating the skin and the hair.

Accordingly, it is possible to use, as active material, conditioning agents for the skin and the hair, such as in particular polymers comprising quaternary ammoniums which may be optionally used in heterocycles (compounds of the quaternium and polyquaternium types, and the like), humectants; fixing (styling) agents which are more particularly chosen from polymers (homo-, co- or terpolymers, for example acrylamide, acrylamide/sodium acrylate, polystyrenesulfonate, and the like), cationic polymers, polyvinylpyrrolidone, polyvinyl acetate, and the like.

It is likewise possible to use colorants; astringents, which can be used in deodorants and which are more particularly aluminum or zirconium salts; antibacterial agents; anti-inflammatory agents, anesthetizing agents, sunscreens, and the like.

There may also be mentioned α- and β-hydroxy acids, such as citric, lactic, glycolic and salicylic acids; dicarboxylic acids, preferably unsaturated and comprising 9 to 16 carbon atoms such as azelaic acid; vitamin C and its derivatives, in particular the glycosylated and phosphated derivatives; biocides, in particular cationic biocides, as active materials suitable in cosmetic and/or dermatological formulations.

In accordance with a particularly advantageous embodiment of the present invention, the internal aqueous phase may comprise at least one additive chosen from salts such as alkali or alkaline-earth metal halides (such as sodium chloride and calcium chloride), or alkali or alkaline-earth metal sulfates (such as magnesium sulfate), or mixtures thereof. The internal aqueous phase may also comprise, as additive, at least one sugar, such as glucose for example, or alternatively at least one polysaccharide, such as in particular dextran, or mixtures thereof. It is obviously possible to have a combination of these various types of additive.

The concentration of salt in the internal aqueous phase, when the latter is present, is more particularly between 0.05 and 1 mol/l, preferably 0.1 to 0.4 mol/l.

The concentration of sugar and/or polysaccharide, if it (they) is (are) used, is such that the osmotic pressure of the internal aqueous phase comprising the sugar and/or the polysaccharide corresponds to the osmotic pressure of an internal aqueous phase comprising 0.05 to 1 mol/l of salt.

In addition, the inverse emulsion of the multiple emulsion has more particularly an aqueous phase/oily phase weight ratio of between 10/90 and 90/10. Preferably, the aqueous phase/oily phase weight ratio is between 30/70 and 80/20.

The inverse emulsion is prepared using conventional methods.

Thus, to cite only one example, there are prepared, on the one hand, a first mixture comprising water, the hydrophilic active material and optionally the additive (salt, sugar, polysaccharide), and, on the other hand, a second mixture comprising the oil, optionally the hydrophobic active material and the surfactant and/or the amphiphilic polymer. The first mixture is then added to the second, with stirring.

In the case where the oily phase is not very viscous (viscosity less than 1 Pa.s), the stirring is preferably vigorous and may be advantageously provided by the use of an apparatus such as Ultra-Turrax®, Microfluidizer, or any high-pressure homogenizer.

In the case where the oily phase is viscous (viscosity greater than or equal to 1 Pa.s, preferably greater than or equal to 5 Pa.s), the stirring may be advantageously carried out by means of a paddle frame.

The preparation of the inverse emulsion is generally carried out at a temperature greater than the melting point of the oily phase. Usually, the temperature for preparing the inverse emulsion is between 20 and 80° C.

The duration of the stirring may be determined without difficulty by persons skilled in the art and depends on the type of apparatus used. It is preferably sufficient to obtain a mean droplet size of between 0.1 and 10 μm, preferably between 0.1 and 5 μm (measured by means of a Horiba granulometer).

The external aqueous phase of the multiple emulsion will now be described.

As indicated above, the external aqueous phase may comprise at least one active material which is solubilized in the external aqueous phase, or in a solubilized form in a solvent miscible with the external aqueous phase, such as methanol, ethanol, propylene glycol, glycerol, or in the form of a divided solid dispersed in said external aqueous phase.

The content of active material, if it is present, is more particularly between 0.1 and 50% by weight of the external aqueous phase, and preferably between 0.1 and 20% by weight of the external aqueous phase.

According to a first possibility, the external aqueous phase further comprises at least one nonionic surfactant and/or at least one nonionic amphiphilic polymer optionally combined with at least one anionic amphiphilic polymer. According to a second possibility, the external aqueous phase comprises at least one anionic amphiphilic polymer optionally combined with at least one anionic surfactant. It should be noted that the Bancroft rule may be applied to the surfactants and polymers used (fraction soluble in the continuous phase is higher than the fraction soluble in the dispersed phase).

Thus, the surfactant and the polymer are preferably chosen from those which simultaneously satisfy the two conditions below:
when they are mixed with the external aqueous phase, at a concentration of between 0.1 and 10% by weight of said phase at 25° C., they exist in the form of a solution in the whole or part of the concentration range indicated;
when they are mixed with the internal oily phase, at a concentration between 0.1 and 10% by weight of said phase and at 25° C., they exist in the form of a dispersion in the whole or part of the concentration range indicated.

More particularly, the external aqueous phase comprises:
at least one nonionic surfactant and/or at least one nonionic amphiphilic polymer optionally combined with at least one anionic surfactant and/or at least one anionic amphiphilic polymer; the total content of nonionic and anionic surfactant(s)/amphiphilic polymer(s) is between 0.5 and 10% by weight, preferably between 1 and 5% by weight, relative to the oily phase or to the inverse emulsion if it is present; the quantity of anionic surfactant and/or amphiphilic polymer represents 0.5 to 5% by weight, preferably 0.5 to 2% by weight, relative to the weight of nonionic surfactant/amphiphilic polymer; or
at least one anionic amphiphilic polymer optionally combined with at least one anionic surfactant; the total content of anionic amphiphilic polymer/surfactant is between 0.5 and 10% by weight, preferably between 1 and 5% by weight, relative to the oily phase or to the inverse emulsion if it is present.

Advantageously, the surfactants and polymers chosen do not give a liquid crystal phase in the aqueous phase.

As regards the nonionic surfactants, polyalkoxylated nonionic surfactants are preferably used.

Advantageously, said nonionic surfactant is chosen from the following surfactants, alone or as a mixture:
alkoxylated fatty alcohols
alkoxylated triglycerides
alkoxylated fatty acids
alkoxylated sorbitan esters
alkoxylated fatty amines
alkoxylated di(1-phenylethyl)phenols
alkoxylated tri(1-phenylethyl)phenols
alkoxylated alkylphenols
   the number of alkoxylated, more particularly ethoxylated and/or propoxylated, units is such that the HLB value is greater than or equal to 10.

As regards the nonionic amphiphilic polymer, a polymer is used which comprises at least two blocks, one of them being hydrophilic, the other hydrophobic.

What was indicated above in the context of the description of the nonionic hydrophilic monomers and of the hydrophobic monomers which can be used for preparing the amphiphilic block polymers entering into the composition of the inverse emulsion remains valid and will not be repeated here; the proportion and the nature of said monomers being such that the resulting polymer satisfies the conditions set out above (Bancroft rule—two conditions).

According to a particular embodiment, the polymer is obtained from hydrophilic monomers chosen from acrylamide and methacrylamide, alone or as a mixture, or in the form of macromonomers; the preferred hydrophobic monomers are esters of acrylic acid with linear or branched C₁-C₄ alcohols such as methyl, ethyl, propyl and butyl acrylate, vinyl esters such as vinyl acetate, styrene, α-methylstyrene.

In the case where said polymer comprises at least three blocks, and more particularly three blocks, the polymer is advantageously linear. In addition, the hydrophilic blocks are present more particularly at the ends.

In the case where the polymers comprise more than three blocks, the latter are preferably in the form of graft or comb polymers.

According to another advantageous embodiment, the amphiphilic polymer comprises polyalkoxylated blocks, and preferably comprises only polyalkoxylated blocks, at least one of which is hydrophobic, the other hydrophilic.

Purely as a guide, said polymers are obtained using in particular anionic ring opening polymerizations.

More particularly, said nonionic polyalkoxylated amphiphilic polymers are chosen from the polymers whose weight-average molar mass is less than or equal to 100 000 g/mol (measured by GPC, polyethylene glycol standard), preferably between 1 000 and 50 000 g/mol, preferably between 1 000 and 20 000 g/mol.

By way of examples of polymers of this type, there may be mentioned, inter alia, polyethylene glycol/polypropylene glycol/polyethylene glycol triblock polymers. Such polymers are well known and are in particular marketed under the trademarks Pluronic (marketed by BASF), Arlatone (marketed by ICI).

Among the suitable anionic surfactants, there may be mentioned, inter alia, alone or as mixtures:
alkyl ester sulfonates, for example of formula R—CH(SO₃M)-COOR′, where R represents a C₈-C₂₀, preferably C₁₀-C₁₆, alkyl radical, R′ a C₁-C₆, preferably C₁-C₈, alkyl radical and M an alkali metal cation (sodium, potassium, lithium), a substituted or unsubstituted ammonium (methyl-, dimethyl-, trimethyl-, tetramethylammonium, dimethylpiperidinium and the like) or a derivative of an alkanolamine (monoethanol-amine, diethanolamine, triethanolamine and the like). There may be mentioned most particularly the methyl ester sulfonates whose radical R is C₁₄-C₁₆; alkyl, more particularly C₉-C₂₀ alkylbenzene sulfonates, primary or secondary alkyl, in particular C₈-C₂₂, alkyl sulfonates, alkylglyceryl sulfonates, sulfonated polycarboxylic acids, such as for example those described in GB 1082179, paraffin sulfonates;
alkyl sulfates for example of formula ROSO₃M, where R represents a C₁₀-C₂₄, preferably C₁₂-C₂₀, alkyl or hydroxyalkyl radical; M representing a hydrogen atom or a cation having the same definition as above, as well as their polyalkoxylated (ethoxylated (EO), propoxylated (PO), or combinations thereof) derivatives, such as for example sodium dodecyl sulfate;
alkyl ether sulfates for example of formula RO(CH₂CH₂O)ₙSO₃M where R represents a C₁₀-C₂₄, preferably C₁₂-C₂₀, alkyl or hydroxyalkyl radical; M representing a hydrogen atom or a cation having the same definition as above, n generally varying from 1 to 4, and their polyalkoxylated (ethoxylated (EO), propoxylated (PO), or combinations thereof) derivatives, such as for example lauryl ether sulfate with n=2;
alkyl amide sulfates for example of formula RCONHR′ OSO₃M where R represents a C₂-C₂₂, preferably C₆-C₂₀, alkyl radical, R′ a C₂-C₃ alkyl radical, M representing a hydrogen atom or a cation having the same definition as above, and their polyalkoxylated (ethoxylated (EO), propoxylated (PO), or combinations thereof) derivatives;
salts of saturated or unsaturated fatty acids, for example such as those in the form of C₈-C₂₄, preferably C₁₄-C₂₀, N-acyl N-alkyllaurates, alkyl isethionates, alkyl succinamates and alkyl sulfosuccinates, monoesters or diesters of sulfosuccinates, N-acylsarcosinates, polyethoxycarboxylates; and
alkyl and/or alkyl ether and/or alkyl aryl ether phosphate esters.

Among the anionic polymers which can be used, there may be mentioned most particularly block, preferably diblock or triblock, polymers obtained by polymerization of at least one anionic hydrophilic monomer, optionally of at least one nonionic hydrophilic monomer, and of at least one hydrophobic monomer.

Here again, the choice of the monomers and their respective proportions are such that the resulting polymer satisfies the two conditions set out above (Bancroft rule).

The nonionic or anionic hydrophilic monomers, or the hydrophobic monomers and their modes of synthesis cited in the context of the description of the amphiphilic polymers entering into the composition of emulsions for which the continuous phase is an oily phase may be used for producing the polymers according to this variant. Reference can therefore be made thereto.

It should be noted that there will be no departure from the context of the present invention by combining one or more surfactants with one or more amphiphilic polymers.

However, according to a preferred embodiment of the invention, the external aqueous phase comprises one or more amphiphilic polymers.

Moreover, there would be no departure from the context of the present invention by using a cationic amphiphilic polymer, in combination with the nonionic surfactant and/or the nonionic amphiphilic polymer cited above.

Among the cationic polymers which can be used in the context of the present invention, there may be mentioned most particularly the cationic derivatives of polysaccharides, such as guar or cellulose derivatives.

It is likewise possible to use cationic polymers functionalized with hydrophobic groups such as C₁-C₁₄, preferably C₂-C₈, alkyl chains optionally having a hydroxyl group. These hydrophobic groups are attached to the principal polymeric chain via ether bonds.

Moreover, and in the case of hydrophobic or nonhydrophobic modified cationic guars, the cationic group is a quaternary ammonium group carrying three radicals, which may or may not be identical, chosen from hydrogen, an alkyl radical comprising 1 to 22 carbon atoms, more particularly 1 to 14, advantageously 1 to 3 carbon atoms. The counter-ion is a halogen, preferably chlorine.

In the case of hydrophobic or nonhydrophobic modified cationic celluloses, the cationic group is a quaternary ammonium group carrying three radicals, which may or may not be identical, chosen from hydrogen, an alkyl radical comprising 1 to 10 carbon atoms, more particularly 1 to 6, advantageously 1 to 3 carbon atoms. The counter-ion is a halogen, preferably chlorine.

Furthermore, the weight ratio of the inverse emulsion in relation to the external aqueous phase in the multiple emulsion is usually between 30/70 and 90/10, preferably between 50/50 and 90/10.

In order to equilibrate the osmotic pressures of the external aqueous phase and of the internal aqueous phase, it is possible to add to the external aqueous phase at least one additive chosen from salts such as alkali or alkaline-earth metal halides (such as sodium chloride or calcium chloride), from alkali or alkaline-earth metal sulfates (such as magnesium sulfate), from sugars (glucose for example), or alternatively from polysaccharides (in particular dextran) or mixtures thereof.

The concentrations of additive (salt, sugar and/or polysaccharide) are such that the osmotic pressures of the external and internal aqueous phases are balanced.

Furthermore, depending on the application for which the emulsion according to the invention is intended, or depending on the nature of the active material, it may be advantageous to adjust the pH of the external aqueous phase by adding a base (sodium hydroxide or potassium hydroxide) or an acid (hydrochloric acid).

By way of illustration, the usual pH range for the external aqueous phase is between 3 and 8, preferably between 5 and 8.

It should be noted that if the internal oily phase has a relatively high viscosity, for example greater than or equal to 1 Pa.s, more particularly greater than or equal to 5 Pa.s, it may be advantageous to add to the external aqueous phase at least one thermothickening polymer.

It is recalled that this type of polymer has the characteristic of giving aqueous solutions whose viscosity increases when the temperature exceeds a certain threshold temperature. More particularly, these polymers are soluble in water at room temperature, and above the threshold temperature, part of the polymer becomes hydrophobic (heat-sensitive part): the polymer thus forms a physical network on a microscopic scale, which results on a macroscopic scale in an increase in viscosity.

According to an advantageous embodiment of the present invention, the thermothickening polymer used is chosen from polymers exhibiting a jump in viscosity between 25 and 80° C. such that the value of the log₁₀(viscosity at 80° C.)/log₁₀(viscosity at 25° C.) ratio is at least equal to at least 1.

The ratio is measured under the following conditions:
The polymer is first of all dissolved in water (dry extract of 4%).
The Theological profile is then measured in a forced stress flow mode, scanning the temperature between 20° C. and 80° C. The configuration used is the flat-cone geometry 4 cm/1 degree. The stress introduced into the program is chosen (in manual mode) such that the gradient at 25° C. is 10 s⁻¹.
The parameter which was selected for characterizing the thermothickening power of the polymer, that is the log₁₀(viscosity at 80° C.)/log₁₀(viscosity at 25°) ratio, represents the rise in viscosity, expressed in decades, from 25 to 80° C. This parameter expresses in other words that the viscosity of the medium at 80° C. is greater than 10ⁿ times the viscosity of the medium at 25° C.; with n integer being between 0 and 5.

In addition to this characteristic, the thermothickening polymer is chosen such that the variation in viscosity is reversible.

Among the thermothickening polymers which can be used, there may be mentioned hydrophobic modified polysaccharides such as carboxymethyl celluloses, methyl celluloses, hydroxyethyl celluloses and hydroxypropyl celluloses.

In the case of this type of polymer, it may be advantageous to use them combined with at least one additional surfactant, chosen from nonionic or anionic surfactants.

Also suitable are synthetic polymers such as polymers based on N-isopropylacrylamide, polymers based on N,N-dimethylaminoethyl methacrylate.

Polymers with a comb structure consisting of a polymeric backbone segment on which are grafted at least two polymeric side segments, which are identical or different, for which either the polymeric backbone segment, or the polymeric side segments possess a lower critical solubility temperature, LCST, of between 25 and 80° C. Preferably, the polymeric side segments are heat-sensitive and are derived from polyoxyalkylenated polymers.

By way of examples of polymers of this type, there may be mentioned in particular polymers prepared from POE-POP-POE polymer triblocks and acrylic acid (respective molar percentages: 2.3%, 97.7%, direct grafting), polymers prepared from a macromonomer of triblocks POE-POP-POE and acrylic acid (respective mol %: 1.6%, 98.4%, copolymerization), polymers prepared from a macromonomer of triblocks POE-POP-POE and acrylic acid (respective mol %: 3%, 97%, copolymerization), polymers prepared from a macromonomer of triblocks POE-POP-POE and acrylic acid (respective mol %: 2%, 98%, copolymerization).

These polymers were the subject of French patent application FR 2 180 422, to which reference may be made for more information on polymers and their production.

The content of thermothickening polymer represents more particularly, when it is present, 0.2 to 10% by weight of the external aqueous phase. Preferably, the content of this polymer represents 1 to 5% by weight of the external aqueous phase.

According to an advantageous variant of the present invention, the external aqueous phase of the multiple emulsion may comprise at least one thickening polymer. This polymer has in particular the effect of avoiding creaming and/or sedimentation of the final emulsion.

By way of illustration, there may be used thickening polymers extracted from plants and optionally modified, such as carrageenans, alginates, carboxymethyl-celluloses, methylcelluloses, hydroxypropylcelluloses, hydroxyethylcelluloses.

It is likewise possible to use thickening polymers such as polysaccharides of animal, plant or bacterial origin; there may be mentioned by way of nonlimiting example xanthan gum, guar and derivatives (such as hydroxypropylguar for example), polydextroses, or combinations thereof.

When it is present, the content of thickening polymer is more particularly between 0.1 and 2% by weight relative to the external aqueous phase, preferably between 0.1 and 0.5% by weight relative to the external aqueous phase. It should be specified that in this concentration range, the thickening polymer is soluble in the aqueous phase.

According to another variant of the invention, the external aqueous phase comprises a dispersed external oily phase. Optionally, said external aqueous phase may also comprise at least one dispersed divided solid.

The external oily phase may additionally comprise at least one hydrophobic active material.

In the case where the external oily phase comprises one or more different hydrophobic active materials of the oily phase, their content represents more particularly less than or equal to 50% by weight of said external oily phase, preferably from 0.5 to 50% by weight, advantageously between 0.5 and 20% by weight.

More particularly, if the external aqueous phase does not comprise an active material which is soluble, solubilized or dispersed in the form of a divided solid, the external oily phase comprises in this case at least one hydrophobic active material. It should be specified that it is not impossible for both the external aqueous and oily phases to each comprise at least one active material.

All that has been stated above regarding the hydrophobic active material optionally present in the internal oily phase remains valid and will not be detailed again now. Likewise, it is recalled that said external oily phase may in itself constitute an active material.

It should be noted that these active materials may be used in the presence of conventional additives in the relevant field of application.

More particularly, and still according to this variant, the external oily phase represents from 1 to 50% by weight of the external aqueous phase, preferably 5 to 25% by weight of the external aqueous phase.

In addition, it is preferable for the size of the droplets of the external oily phase to be at most of the same order of magnitude as that of the inverse emulsion dispersed in the external aqueous phase.

As for the possibility of using a solid dispersed in the external aqueous phase, all the solids used in the various types of cosmetic and/or dermatological formulations may be suitable. Preferably, the size of this or these dispersed solids is close to or smaller than that of the droplets of the inverse emulsion.

In the case where the dispersed solid is present, its content represents more particularly 1 to 50% by weight of the external aqueous phase, preferably 5 to 25% by weight.

The preparation of the multiple emulsion may be carried out according to any known method.

By way of example, the procedure may be carried out in the following manner. In a first instance, the external aqueous phase is prepared by mixing the surfactant and/or the amphiphilic polymer, optionally the active material if it exists in a form soluble in the aqueous phase or solubilized in a solvent miscible with this phase, optionally the thermothickening polymer, and the water.

Preferably, the water and the surfactant and/or the amphiphilic polymer are first of all mixed, with stirring, and then optionally the active material and the thermothickening polymer and optionally the additive (salt/sugar/polysaccharide).

This operation usually takes place between 20 and 80° C.

The external aqueous phase may be optionally allowed to stand for 1 to 12 hours at room temperature.

The preparation of the multiple emulsion itself is then carried out by adding the inverse emulsion to the external aqueous phase. It should be noted that, advantageously, part of the external aqueous phase is set aside so as to be used for the preparation of the simple emulsion comprising the external oily phase dispersed in the external aqueous phase, in the case where such an emulsion is present.

The preparation of the multiple emulsion is preferably carried out with stirring, while slowly adding at the beginning the inverse emulsion.

The stirring may be carried out by means of a paddle frame. Typically, the stirring speed is relatively slow, of the order of 400 rpm.

According to the variant where the multiple emulsion is mixed with a simple oil-in-water emulsion, said emulsion composed of the dispersed external oily phase is advantageously added to the same external aqueous phase as that used for the multiple emulsion. It should be noted however that the use of different external aqueous phases is not excluded insofar as there is no destabilization of the mixed emulsion. Quite obviously, the quantities of external aqueous phase introduced with the inverse and simple emulsions are such that the proportions by weight of each of the phases satisfy the conditions explicitly stated above for the mixed emulsion.

The production of the direct emulsion is carried out according to any known method, by mixing, with stirring, the two phases: the external oily phase where appropriate, the hydrophobic active material and the external aqueous phase comprising the surfactant and/or the amphiphilic polymer, optionally the soluble or solubilized active material and optionally the thermothickening polymer.

In the case where the external aqueous phase comprises a dispersed solid, the production of the multiple emulsion may be carried out as indicated in the first case, and then said dispersed solid is added to the external aqueous phase.

The mean size of the droplets of the multiple emulsion varies advantageously between 5 and 50 μm (Horiba).

Finally, if the multiple emulsion comprises a thickening agent, the latter is very advantageously incorporated once the mixed emulsion has been obtained, that is to say once all the other ingredients have been added.

The mixed emulsion which has just been detailed may be used as a constituent element of cosmetic and/or dermatological formulations.

The content of mixed emulsion is preferably such that the total content of active material(s) present in the aqueous and oily phases, in the cosmetic and/or dermatological formulation is between 0.01 and 10% by weight of said formulation.

The cosmetic and/or dermatological formulations which are the subject of the present invention may be formulated as a large number of types of product for the skin, the hair, the eyelashes and/or the nails, conditioners, formulations for hair styling or for facilitating combing of the hair, lotions for the hands and the body, products regulating skin moisturization, skincare milks, makeup-removing compositions, depilatory products, creams or lotions for protecting against sunlight and ultraviolet radiation, care creams, antiacne preparations, makeup formulations of the mascara, foundation or nail varnish type, products intended to be applied to the lips, and the like.

The cosmetic and/or dermatological formulations, in addition to the mixed emulsions, comprise conventional additives in the field.

It is thus possible to incorporate into the cosmetic and/or dermatological formulation, in the form of dispersions or solutions, bactericidal or fungicidal agents so as to improve disinfection of the skin, such as for example triclosan; antidandruff agents, such as in particular zinc pyrithione or octopyrox; insecticidal agents such as natural or synthetic pyrethroids.

The cosmetic and/or dermatological formulations may also contain agents for protecting the skin and/or the hair against attacks by sunlight and UV radiation. Thus, the compositions may comprise sunscreens which are chemical compounds which strongly absorb UV radiation, such as the compounds authorized in the European directive No. 76/768/EEC, its annexes and subsequent amendments of this directive, in particular organic particles such as zinc oxide, titanium dioxide or cerium oxides in powdered form or in the form of colloidal particles, alone or as a mixture. These powders may be optionally surface-treated in order to increase the efficiency of their anti-UV action and to facilitate their incorporation into cosmetic formulations or to inhibit surface photoreactivity.

Said formulations may also contain fixing resins.

These fixing resins, when they are present, are generally at concentrations of between 0.01 and 10%, preferably between 0.5 and 5%.

The fixing resins entering into the cosmetic compositions are more particularly chosen from the following resins:
methyl acrylate/acrylamide copolymers, polyvinyl methyl ether/maleic anhydride copolymers, vinyl acetate/crotonic acid copolymers, octylacryl-amide/methyl acrylate/butylaminoethyl methacrylate copolymers, polyvinylpyrrolidones, polyvinyl-pyrrolidone/methyl methacrylate copolymers, polyvinylpyrrolidone/vinyl acetate copolymers, polyvinyl alcohols, polyvinyl alcohol/crotonic acid copolymers, polyvinyl alcohol/maleic anhydride copolymers, hydroxypropylcelluloses, hydroxypropylguars, sodium polystyrene sulfonates, polyvinylpyrrolidone/ethyl methacrylate/meth-acrylic acid terpolymers, poly(methyl vinyl ether/maleic acid) monomethyl ethers, polyvinyl acetates grafted onto polyoxyethylenated trunks (EP-A-219 048),
copolyesters derived from terephthalic and/or isophthalic and/or sulfoisophthalic acid, anhydride or diester and from a diol, such as:
   polyester copolymers based on ethylene terephthalate and/or propylene terephthalate and/or polyoxyethylene terephthalate units (U.S. Pat. No. 3,959,230, U.S. Pat. No. 3,893,929, U.S. Pat. No. 4,116,896, U.S. Pat. No. 4,702,857, U.S. Pat. No. 4,770,666);
   sulfonated polyester oligomers obtained by sulfonation of an oligomer derived from ethoxylated allyl alcohol, from dimethyl terephthalate and from 1,2-propylenediol (U.S. Pat. No. 4,968,451);
   polyester copolymers derived from dimethyl terephthalate, isophthalic acid, dimethyl sulfoisophthalate and ethylene glycol (EP-A-540374);
   copolymers comprising polyester units derived from dimethyl terephthalate, isophthalic acid, dimethyl sulfoisophthalate and ethylene glycol and polyorganosiloxane units (FR-A-2 728 915);
   sulfonated polyester oligomers obtained by condensation of isophthalic acid, dimethyl sulfosuccinate and diethylene glycol (FR-A-2 236 926);
   polyester copolymers based on propylene terephthalate and polyoxyethylene terephthalate units terminated with ethyl or methyl units (U.S. Pat. No. 4,711,730) or polyester oligomers terminated with alkylpolyethoxy groups (U.S. Pat. No. 4,702,857) or anionic sulfopolyethoxy groups (U.S. Pat. No. 4,721,580), or sulfoaroyls (U.S. Pat. No. 4,877,896);
   polyesters-polyurethanes obtained by reacting a polyester obtained from adipic acid and/or terephthalic acid and/or sulfoisophthalic acid and a diol, with a prepolymer containing terminal isocyanate groups obtained from a polyoxyethylene glycol and a diisocyanate (FR-A-2 334 698);
ethoxylated monoamines or polyamines and ethoxylated amine polymers (U.S. Pat. No. 4,597,898, EP-A-11 984).

Preferably, the fixing resins are chosen from polyvinylpyrrolidone (PVP), polyvinylpyrrolidone and methyl methacrylate copolymers, polyvinylpyrrolidone and vinyl acetate (VA) copolymer, polyethylene glycol terephthalate/polyethylene glycol copolymers, polyethylene glycol terephthalate/polyethylene glycol/sodium polyisophthalate sulfonate copolymers, and mixtures thereof.

These fixing resins are preferably dispersed or solubilized in the chosen vehicle.

The cosmetic and/or dermatological formulations may also contain polymeric derivatives exerting a protective function.

These polymeric derivatives may be present in quantities of the order of 0.01-10% by weight of the cosmetic and/or dermatological formulation, and preferably about 0.1-5% by weight, and most particularly of the order of 0.2-3% by weight.

These agents may be chosen in particular from:
nonionic cellulose derivatives such as cellulose hydroxyethers, methylcellulose, ethylcellulose, hydroxypropylmethylcellulose, hydroxybutylmethylcellulose;
polyvinyl esters grafted onto polyalkylenated trunks such as polyvinyl acetates grafted onto polyoxyethylenated trunks (EP-A-219 048);
polyvinyl alcohols.

The cosmetic and/or dermatological formulations may also comprise plasticizers.

Said agents, when they are present, may represent between 0.1 and 20% by weight of the formulation, preferably from 1 to 15% by weight.

Among the plasticizers which are particularly useful, there may be mentioned adipates, phthalates, isophthalates, azelates, stearates, silicone copolyols, glycols, castor oil, or mixtures thereof.

It is also possible to incorporate into said cosmetic and/or dermatological formulations humectants, among which are, inter alia, glycerol, sorbitol, urea, collagen, gelatin, Aloe vera, hyaluronic acid or water-soluble volatile solvents such as ethanol or propylene glycol whose contents may be up to 60% by weight of the composition.

To further reduce irritation or attack on the scalp, it is also possible to add water-soluble or water-dispersible polymers such as collagen or certain nonallergenic derivatives of animal or plant proteins (hydrolysates of wheat proteins for example), natural hydrocolloids (guar, carob or tara gum and the like) or hydrocolloids derived from fermentation processes, and derivatives of these polycarbohydrates such as nonionic modified celluloses such as for example hydroxyethylcellulose, or anionic modified celluloses such as carboxymethylcellulose; guar or carob derivatives such as their nonionic derivatives (for example hydroxypropylguar) or the anionic derivatives (carboxymethylguar and carboxymethylhydroxypropylguar).

To these compounds there may be added in combination organic powders or particles such as calcium carbonate, sodium bicarbonate, calcium dihydrogen phosphate, organic oxides in powdered form or in colloidal form (particles having a size of less than or of the order of one micrometer, sometimes a few tens of nanometers) such as titanium dioxide, silica, aluminum salts generally used as antiperspirants, kaolin, talc, clays and derivatives thereof, and the like.

Preservatives such as methyl, ethyl, propyl and butyl esters of p-hydroxybenzoic acid, sodium benzoate, GERMABEN® or any chemical agent which avoids the proliferation of bacteria or of molds and which is traditionally used in cosmetic and/or dermatological formulations may also be introduced into the present formulations, generally in an amount of 0.01 to 3% by weight of the formulation.

The quantity of these products is usually adjusted to avoid any proliferation of bacteria, molds or yeasts in the cosmetic compositions.

To these ingredients there may be added, if necessary, and with the aim of increasing comfort during the use of the formulation by the consumer, one or more perfumes, coloring agents, among which there may be mentioned the products described in annex IV (“List of colouring agents allowed for use in cosmetic products”) of the European directive No. 76/768/CEE of 27 Jul. 1976 called cosmetics directive, and/or opacifying agents such as pigments.

Although not obligatory, the formulation may also contain gelling or viscosity promoting polymers so as to adjust the texture of the formulation, such as cross-linked polyacrylates (Carbopol marketed by Goodrich), noncationic derivatives of cellulose such as hydroxypropylcellulose, carboxymethylcellulose, guars and their nonionic derivatives, xanthan gum and its derivatives, used alone or in combination, or the same compounds, generally in the form of water-soluble polymers modified by hydrophobic groups covalently linked to the polymer backbone as described in patent WO 92/16187.

The cosmetic and/or dermatological formulations may also contain polymeric dispersing agents in a quantity of the order of 0.1-7% by weight, in order to control the calcium and magnesium hardness, agents such as:
water-soluble salts of polycarboxylic acids having a weight-average molecular mass of the order of 2000 to 100 000 g/mol, obtained by polymerization or copolymerization of ethylenically unsaturated carboxylic acids such as acrylic acid, maleic acid or anhydride, fumaric acid, itaconic acid, aconitic acid, mesaconic acid, citraconic acid, methylenemalonic acid, and most particularly polyacrylates having a weight-average molecular mass of the order of 2000 to 10 000 g/mol (U.S. Pat. No. 3,308,067), copolymers of acrylic acid and maleic anhydride having a weight-average molecular mass of the order of 5000 to 75 000 g/mol (EP-A-66 915);
polyethylene glycols having a weight-average molecular mass of the order of 1000 to 50 000 g/mol.

It is also possible to advantageously add to these formulations metal sequestrants, more particularly those sequestering calcium such as citrate ions.

A concrete but nonlimiting example of the invention will now be presented.

### EXAMPLE

### 1/Inverse Emulsion

Composition of the Inverse Emulsion:

* 30% of aqueous phase:
14% of lactic acid (% by weight of a 0.1M solution expressed relative to the weight of aqueous phase)
86% of NaCl (% by weight of a 0.1M solution expressed relative to the weight of aqueous phase)

* 70% of oily phase (soybean oil and surfactant) comprising 5% of surfactant (Arlacel P135; ICI—Uniquema (*); % expressed by weight relative to the weight of aqueous phase).

(*) Arlacel P 135: polyhydroxystearate—PEG—polyhydroxystearate; HLB=5-6; Mw=5000 g/mol)

Preparation of the Inverse Emulsion:

100 g of inverse emulsion are prepared. comprising 70 g of internal oily phase and 30 g of internal aqueous phase.

On the one hand, 4.2 g of a 0.1M lactic acid solution are mixed with 25.8 g of a 0.1M NaCl solution.

On the other hand, 1.5 g of Arlacel P135 are mixed with 68.5 g of soybean oil. Before mixing, the soybean oil and the Arlacel were placed in an oven at 75° C.

The internal aqueous phase is then added to the internal oily phase, with Ultraturrax stirring at 9500 rpm. The mixing is carried out at 75° C.

### 2/Multiple Emulsion

Composition of the Multiple Emulsion:

* 50% of inverse emulsion

* 50% of dry extract in external aqueous phase containing:
2% of Arlatone F127G (*) (ICI—Uniquema; % by weight expressed relative to the weight of the inverse emulsion);
3.6% of glucose (% by weight expressed relative to the weight of external aqueous phase);
1% of Rhodopol 23 (**) (Rhodia Chimie; % by weight expressed relative to the weight of external aqueous phase).

(*) Arlatone F127G: HO(CH₂CH₂O)ₓ(OCH(CH₃)CH₂O)_{y}(CH₂CH₂O)_{z}H with verification of the following inequality: 82<x+z<90 and the polymer comprises 7 PO units per 1 mol of product).

(**) Rhodopol 23: xanthan gum.

Preparation of the Multiple Emulsion:

Preparation of the External Aqueous Phase:

40 g of external aqueous phase are prepared by weighing 20 g of a solution of Rhodopol 23 at 2% and 20 g of a solution of Arlatone F F127G at 4% and of glucose at 7.2% are added.

The whole is mixed with a paddle frame at 200 revolutions/minute for 5 minutes.

Preparation of the Multiple Emulsion:

50 g of the inverse emulsion obtained from point 1/ are introduced dropwise, with stirring using a paddle frame at 400 rpm, into the external aqueous phase, at room temperature.

The stirring is maintained for another 10 minutes.

0.5 g of formaldehyde is then added and then the mixture is homogenized for 5 minutes at 400 revolutions/minute.

### 3/Direct Retinol Emulsion

Composition of the Direct Emulsion:

* 32.5% of oily phase comprising 4.8% by weight of retinol (*) (BASF; % by weight expressed relative to the weight of aqueous phase);

* 67.5% of external aqueous phase containing:
2% of Arlatone F127G (% by weight expressed relative to the weight of aqueous phase);
5.3% of glucose (% by weight expressed relative to the weight of aqueous phase);
1.48% of Rhodopol 23 (% by weight expressed relative to the weight of aqueous phase);
10% of Alkamuls T85 (**) (Rhodia Chimie; % by weight expressed relative to the weight of oily phase).

(*) retinol: 10% solution in miglyol.

(**) Alkamuls T85: sorbitan ester comprising 20 ethoxylated units.

Preparation of the Direct Emulsion:

30 g of direct emulsion comprising 9.75 g of retinol solution and 20.25 g of aqueous phase comprising 1 g of Alkamuls T85, 12 g of a solution of Rhodopol at 2.5%, 3.75 g of a solution of Arlatone at 15.6%, and 3.5 g of a solution of glucose at 30% are prepared.

The emulsion is obtained in the following manner:

9.75 g of retinol solution containing 1 g of Alkamuls T85 are mixed with stirring using a paddle frame at 500 revolutions/minute.

3.75 g of Arlatone F127G solution are introduced dropwise, with stirring using a paddle frame at 500 revolutions/minute. At the end of the addition, the stirring is maintained for 10 minutes.

Stirring is then carried out with Ultra-turrax at 9500 revolutions/minute (5 minutes) and then at 20 500 revolutions/minute (2 minutes). During this stirring, the emulsion is cooled.

The glucose solution is then introduced into the retinol emulsion with stirring using a paddle frame at 500 revolutions/minute, and then the Rhodopol 23 solution is introduced all at once while mixing with a spatula.

Homogenization is finally performed with an Ultra-turrax at 9500 revolutions/minute.

### 4/Mixed Emulsion

10 g of retinol emulsion are introduced all at once into 90 g of multiple emulsion.

The medium is gently mixed with a spatula and then homogenized with a paddle frame at 200 rpm for 3 minutes.

A stable mixed emulsion is obtained comprising 0.5% by weight of retinol relative to the total water.

1-18. (canceled)

19. A cosmetic and/or dermatological formulation comprising at least two active materials in a multiple emulsion consisting of an internal aqueous phase dispersed in an internal oily phase, the whole being dispersed in an external aqueous phase; the multiple emulsion being optionally mixed with a simple emulsion consisting of an external oily phase dispersed in an external aqueous phase:
the internal oily phase comprising at least one nonionic surfactant and/or at least one amphiphilic polymer and optionally at least one hydrophobic active material;
the external aqueous phase comprising at least one nonionic surfactant and/or at least one nonionic amphiphilic polymer optionally combined with at least one anionic amphiphilic polymer or comprising at least one anionic amphiphilic polymer optionally combined with at least one anionic surfactant;
at least one hydrophilic active material which is present in the internal aqueous phase; and
at least one active material which is present in a soluble or solubilized form or in the form of a solid dispersed in the external aqueous phase, or which is present in the external oily phase.

20. The formulation as claimed in claim 19, wherein the surfactant and/or the polymer present in the internal oily phase simultaneously satisfy the two conditions below:
when they are mixed with the internal oily phase, at a concentration between 0.1 and 10% by weight of said phase at 25° C., they are in the form of a solution in the whole or part of the concentration range indicated; and
when they are mixed with the internal aqueous phase, at a concentration between 0.1 and 10% by weight of said phase and at 25° C., they are in the form of a dispersion in the whole or part of the concentration range indicated.

21. The formulation as claimed in claim 19, wherein the quantity of nonionic surfactant and/or nonionic amphiphilic polymer represents 2 to 10% by weight of the internal aqueous phase.

22. The formulation as claimed in claim 19, wherein the content of hydrophilic active material present in the internal aqueous phase is between 0.1 and 50% by weight of the internal aqueous phase.

23. The formulation as claimed in claim 19, wherein the inverse emulsion has an aqueous phase/oily phase weight ratio of between 10/90 and 90/10.

24. The formulation as claimed in claim 19, wherein the internal aqueous phase further comprises at least one additive selected from the group consisting of alkali metal halides, alkaline-earth metal halides, alkali metal sulfates, alkaline-earth metal sulfates, sugars, and polysaccharides.

25. The formulation as claimed in claim 24, wherein the concentration of the halide or sulfate additive in the internal aqueous phase is between 0.05 and 1 mol/l, and/or the concentration of sugar or polysaccharide is such that the osmotic pressure of the internal aqueous phase comprising the sugar and/or the polysaccharide corresponds to the osmotic pressure of an internal aqueous phase comprising 0.05 to 1 mol/l of salt.

26. The formulation as claimed in claim 19, wherein the surfactant and/or the polymer present in the external aqueous phase satisfy the two conditions below:
when they are mixed with the external aqueous phase, at a concentration between 0.1 and 10% by weight of said phase at 25° C., they are in the form of a solution in the whole or part of the concentration range indicated; and
when they are mixed with the internal oily phase, at a concentration between 0.1 and 10% by weight of said phase and at 25° C., they are in the form of a dispersion in the whole or part of the concentration range indicated.

27. The formulation as claimed in claim 19, wherein the external aqueous phase further comprises at least one thermothickening polymer.

28. The formulation as claimed in claim 27, wherein the thermothickening polymer is a polymer exhibiting a rise in viscosity between 25 and 80° C. such that the value of the log₁₀(viscosity at 80° C.)/log₁₀(viscosity at 25° C.) ratio is at least equal to at least 1.

29. The formulation as claimed in claim 27, wherein the thermothickening polymer content is between 0.2 and 10% by weight of the external aqueous phase.

30. The formulation as claimed in claim 19, wherein the external aqueous phase of the multiple emulsion further comprises at least one thickening polymer.

31. The formulation as claimed in claim 30, wherein the content of thickening polymer is between 0.1 and 2% by weight relative to the external aqueous phase.

32. The formulation as claimed in claim 19, having a weight ratio of inverse emulsion in relation to the external aqueous phase in the multiple emulsion of between 30/70 and 90/10.

33. The formulation as claimed in claim 19, wherein the osmotic pressures of the external aqueous phase and of the internal aqueous phase are balanced by adding to the external aqueous phase at least one additive being alkali or alkaline-earth metal halides, alkali or alkaline-earth metal sulfates, or sugars or alternatively polysaccharides.

34. The formulation as claimed in claim 33, wherein the dispersed external oily phase represents 1 to 50% by weight of the external aqueous phase.

35. The formulation as claimed in claim 19, wherein the external aqueous phase further comprises at least one dispersed solid.

36. The formulation as claimed in claim 35, wherein the dispersed solid represents 1 to 50% by weight of the external aqueous phase.
